(19) United States
(12) Patent Application Publication
(10) Pub. No.: US 2009/0326300 A1
(43) Pub. Date: Dec. 31, 2009

(54) PROCESS FOR CRACKING TERT-ALKYL ETHERS THAT USE A MESOSTRUCTURED HYBRID ORGANIC-INORGANIC MATERIAL

(75) Inventors: Vincent COUPARD, Valencin (FR); Alexandra Chaumonnot, Lyon (FR)

Correspondence Address: MILLEN, WHITE, ZELANO & BRANIGAN, P.C., 2200 CLARENDON BLVD., SUITE 1400, ARLINGTON, VA, 22201, US

(73) Assignee: IFP, Rueil Malmaison Cedex (FR)

(21) Appl. No.: 12/469,821

(22) Filed: May 21, 2009

(30) Foreign Application Priority Data
May 23, 2008 (FR) 08/02.800

(51) Int. Cl. C07C 1/20; B01J 31/02

(52) U.S. Cl. 585/640; 502/158

(57) ABSTRACT

A process for cracking tert-alkyl ether(s) selected from among tert-amyl methyl ether (TAME) and ethyl tert-amyl ether (ETAE) for the production of tertiary olefins comprising bringing said tert-alkyl ether(s) into contact with at least one catalyst that is formed by at least one mesostructured hybrid organic-inorganic material that consists of at least two spherical elementary particles, whereby each of said spherical particles consists of a mesostructured matrix with a silicon oxide base to which are linked organic groups with acid terminal reactive functions, said groups representing less than 20 mol % of said matrix that is present in each of said spherical elementary particles, which have a maximum diameter of between 50 nm and 200 μm.

### TECHNICAL FIELD OF THE INVENTION

This invention relates to the field of the decomposition of tert-alkyl ether(s) by cracking for the purpose of selectively producing high-purity tertiary olefins. The tert-alkyl ethers that are targeted by this invention are tert-amyl methyl ether (TAME) and ethyl tert-amyl ether (ETAE). More specifically, this invention relates to a process for cracking tert-alkyl ether(s) selected from among tert-amyl methyl ether (TAME) and ethyl tert-amyl ether (ETAE) for the production of tertiary olefins comprising bringing said tert-alkyl ether(s) into contact with at least one catalyst that is formed by at least one mesostructured hybrid organic-inorganic material.

### PRIOR ART

The process for decomposition of tert-alkyl ethers into tertiary olefins has been known for a long time, as, for example, the patent application EP-A-0 0 68 785 (1982) shows.

Various acidic solids can be used as catalysts for this reaction.

A first family of acid catalysts that are used consists of mineral solids based on alumina, silica or aluminosilicate. Thus, the patent FR-A-2 291 958 relates to a process for decomposition of TAME or ETAE respectively into isoamylenes and methanol or into isoamylenes and ethanol, with use of catalysts selected from among salts, oxides, or complexes of tetravalent uranium, able to be supported on an alumina that has a Lewis acidity. The patent WO-A-91/01 804 describes the production of isoamylenes from TAME via the use of a clay-based catalyst that is treated with an acid that is selected from among hydrofluoric acid, hydrochloric acid, or a mixture of the two. The patent U.S. Pat. No. 5,227,564 describes the decomposition of TAME, in vapor phase and in the presence of a catalyst that contains an aluminosilicate-type zeolite, and the patents EP-A-0 589 557 and U.S. Pat. No. 4,536,605 describe the use of a catalyst that is based on a calcined aluminosilicate. The patent U.S. Pat. No. 5,171,920 describes the process for obtaining at least one tertiary olefin by decomposition of the corresponding ether, either TAME or ETAE, with a catalyst that consists of silica that is modified by the addition of at least one element such as Li, Cs, Mg, Ca or La, for example. Such solids are not very active due to a lack of acidity, and they have a mediocre stability over time. These various catalysts that are based on alumina, silica or aluminosilicate require the addition of water so as to improve the recovery of alcohol and to prevent the secondary reaction of the corresponding formation of dialkyl ether (DME in the case of methanol, for example). This is described in particular in the patents GB-A-1 165 479 and EP-A-0 589 557. However, the presence of water lowers the activity of the catalyst by lowering its acidity (see in particular the patent GB-A-1 165 479) and can then make it necessary to operate at a higher temperature, which can be harmful to the service life of the catalyst. In addition, the presence of water causes an additional secondary reaction. Actually, the water reacts with tertiary olefins to form an alcohol (2-methyl-butan-2-ol formation in the case of isoamylene, for example). According to this process, a loss of the yield in tertiary olefins is noted.

A second family of catalysts comprising acidic functional organic groups can also be used. Thus, the patent U.S. Pat. No. 5,095,164 describes a process for decomposition of the tert-alkyl ethers, such as TAME or ETAE, by using ion exchange resins, for example sulfonated styrene-divinylbenzene resins. It thus is possible to cite the resin Amberlyst 15® of RHOM & HAAS or the resin M-31 OE that is marketed by DOW CHEMICAL. The patent U.S. Pat. No. 4,447,668 also discloses an ion exchange resin for producing isoamylenes and diisoamylenes from the separation from TAME. One of the major drawbacks of the resins cited above is the impossibility of using them at high temperature, more specifically above 120° C. Actually, at high temperature, these resins lose sulfonic groups and therefore lose their activity and/or their acidity at least in part. However, the reactions for decomposition of the ethers are endothermic; the thermodynamic equilibrium of the reaction is therefore shifted even more toward the production of the olefin since the temperature is high. Thus, an operating temperature that is limited to 120° C. is reflected by a low conversion of the ether and limited by the laws of thermodynamics.

For decades, mesostructured hybrid organic-inorganic (MHOI) materials have been developed.

The new synthesis strategies that make it possible to obtain materials with well-defined porosity in a very broad range, extending from microporous materials to macroporous materials by passing through materials with hierarchized porosity, i.e., having pores of several sizes, have undergone a very broad development within the scientific community since the mid-1990s (G. J. de A. A. Soler-illia, C. Sanchez, B. Lebeau, J. Patarin, Chem. Rev., 2002, 102, 4093). It is known to obtain materials whose pore size is well controlled. In particular, the development of synthesis methods called “soft chemistry” led to the low-temperature processing of mesostructured materials. The soft chemistry methods essentially consist in bringing inorganic precursors, in aqueous solution or in polar solvents, into the presence of an ionic or neutral structuring agent, generally a molecular or supramolecular surfactant. The monitoring of the electrostatic interactions or by hydrogen bonds between the inorganic precursors and the structuring agent, jointly linked to hydrolysis reactions/condensation of the inorganic precursor, leads to a cooperative assembly of the organic and inorganic phases that generate micellar aggregates of surfactants of uniform and controlled size within an inorganic matrix. The release of the porosity is then obtained by elimination of the surfactant, whereby the latter is conventionally carried out by processes of chemical extraction or by heat treatment.

Based on the nature of the inorganic precursors and the structuring agent that is used as well as operating conditions that are imposed, several families of mesostructured materials have been developed. For example, the M41S family initially developed by Mobil (J. S. Beck, J. C. Vartuli, W. J. Roth, M. E. Leonowicz, C. T. Kresge, K. D. Schmitt, C. T.-W. Chu, D. H. Olson, E. W. Sheppard, S. B. McCullen, J. B. Higgins, J. L. Schlenker, J. Am. Chem. Soc., 1992, 114, 27, 10834) consists of mesoporous materials that are obtained via the use of ionic surfactants such as quaternary ammonium salts that have a generally hexagonal, cubic or lamellar structure, pores of uniform size in a range of 1.5 to 10 nm, and amorphous walls with a thickness on the order of 1 to 2 nm. Below, structuring agents of a different chemical nature have been used, such as block copolymer-type amphiphilic macromolecules, whereby the latter lead to mesostructured materials that have a structure that is generally hexagonal, cubic or lamellar, pores of uniform size in a range of 4 to 50 nm, and amorphous walls with a thickness in a range of 3 to 7 nm (families of SBA, MSU, etc.).

The formation of a mesostructured inorganic network passes through a precise monitoring of each of the unit stages of the synthesis. In particular, the chemical composition of the initial solution is a key parameter since the nature and the concentration of each of the reagents and solvents will act on the kinetics of hydrolysis—condensation of various inorganic precursors—and influence the nature and the force of interactions brought into play between the organic and inorganic phases during the self-assembly process. Another crucial stage of the synthesis is the destabilization of this initial solution that will initiate the joint phenomena of self-organization of the structuring agent and hydrolysis—condensation of the inorganic precursors. This destabilization of the initial solution can be the result of chemical phenomena (precipitation, gelling) or physical phenomena (evaporation, temperature). To date, the mesostructured solids most often studied have been obtained according to the methods of synthesis by precipitation (MCM, SBA, MSU). Generally, the synthesis of these materials that are obtained by precipitation requires a curing stage in an autoclave, and all of the reagents are not integrated into products of stoichiometric quantity since they can be found in the supernatant. Based on the structure and the degree of organization desired for the final mesostructured material, these syntheses may take place in an acidic environment (pH≦1) (WO 99/37705) or in a neutral environment (WO 96/39357), whereby the nature of the structuring agent that is used also plays a dominant role. The thus obtained elementary particles do not come in a uniform shape and are generally characterized by a size of greater than 500 nm. Less frequently, mesostructured materials can also be obtained by evaporation of solvents from dilute reagent solutions, whereby this process is usually referred to as “Self-Assembly Caused by Evaporation.” In this case, the principle consists in starting from a dilute reagent solution with a concentration in a structuring agent that is generally less than the critical micellar concentration (Cmc). The gradual evaporation of solvents from the solution leads to a concentration of all of the reagents until the concentration in structuring agent reaches the Cmc and causes the self-assembly of the structuring agent together with the formation of the mesostructured matrix. Compared to the precipitation method, the evaporation method has the advantage of allowing a better monitoring of the hydrolysis—condensation of reagents—to preserve the exact stoichiometry that is defined for the initial solution, and to obtain the desired materials under various morphologies such as films, powders that consist of spherical particles, fibers, etc. Among the techniques by evaporation, we will cite in particular the immersion deposition technique, known by one skilled in the art under the name of “dip-coating” technique, which leads to the formation of mesostructured films by deposition on a substrate (WO 99/15280; A. Brunet-Bruneau, A. Bourgeois, F. Cagnol, D. Grosso, C. Sanchez, J. Rivory, Thin Solid Films, 2004, 656, 455) as well as the aerosol technique that leads to the formation of perfectly spherical nanoparticles after atomization of the initial solution (C. J. Brinker, Y. Lu, A. Sellinger, H. Fan, Adv. Mater., 1999, 11, 7: S. Areva, C. Boissiere, D. Grosso, T. Asakawa, C. Sanchez, M. Linden, Chem. Com., 2004, 1630). It should be noted that obtaining a mesostructured matrix is in general assisted during the immersion deposition technique owing to the presence of the substrate as an anchoring point in the formation of the material relative to the aerosol technique at the end of which a powder is directly obtained.

The extrapolation from a synthesis mode that is carried out by the immersion deposition technique to an aerosol mode is therefore not direct. The aerosol process offers the advantage of allowing the synthesis of materials in an economic and continuous way in the form of powders that can be used as is or after shaping in the industry.

Within the framework of the development of new materials, obtaining hybrid organic-inorganic (MHOI) materials that combine the properties of each of the two phases is of very great advantage (P. Gomez Romero, C. Sanchez (eds.), “Functional Hybrid Materials,” WILEY-VCH, 2004; C. Sanchez, B. Jullian, P. Belleville, M. Popall, J. Mater. Chem., 2005, 15 (35-36), 3559). To date, several synthesis methods lead to the formation of these hybrid materials. In the particular case of interactions of a covalent nature between the organic part and the inorganic part, two synthesis modes are usually encountered:
The direct synthesis that consists in directly incorporating the organic function during the sol-gel synthesis of an inorganic solid by using a metal organic alkoxide precursor, and
The synthesis by post-treatment that consists in obtaining, in a first stage, an inorganic solid and in functionalizing the surface, during a second stage, by reacting a metal organic alkoxide with the surface hydroxyl groups.

The first method that is cited offers the advantage of allowing the incorporation of high contents of organic fragments compared to the post-treatment technique that is limited by the surface condition of the solid that is initially formed. In return, since the organic part is incorporated at the same time that the processing of the inorganic framework is done, the organic sites are not totally accessible. The production of mesostructured MHOI by use of a suitable metal organic alkoxide precursor leads to the formation of a mesostructured hybrid network in which the organic fragments come to be positioned at the walls of the mesopores. The location of the organic part on the surface of the mesopores combined with the mesostructure of the framework promotes the accessibility to the organic sites. The first mesostructured MHOI were obtained in 1996 via the precipitation technique (S. L. Burket, S. D. Sims, S. Mann, Chem. Comm., 1996, 1367). More recently, hybrid organic-inorganic films were obtained by “dip-coating,” whereby the matrix is essentially silicic, and the incorporated organic fragments have a variable nature: carbon-containing alkyl chains, fluorinated alkyl chains, alkyl chains that carry terminal reactive functions—thiol, amine, dinitrophenyl, etc. (U.S. Pat. No. 6,387,453).

Rare examples deal with MHOI processing by an aerosol. A first example deals with the incorporation in the framework itself of the silicic inorganic mesostructured matrix of an organic fragment by use of a particular precursor (OR)₃Si—R′—Si(OR)₃ with R′=—(CH₂)ₙ—, phenyl, vinyl. In this particular case, the organic fragment is an integral part of the framework and is therefore not “hanging” in the mesopores (US 00/46682, 2002). A second example deals with a mesostructured MHOI that is obtained via the use of the organoalkoxysilane precursor (OEt)₃Si—CH₃, whereby the corresponding solid is characterized by the presence of methyl groups that are located on the walls of the pores of the mesostructure.

Obtaining the mesostructured MHOI by an aerosol characterized by organic fragments that carry accessible reactive terminal functions (acid-basic properties, adsorption properties, etc.), beyond simple alkyl chains, has never been reported to our knowledge. This is probably explained by the difficulty of monitoring interactions between the various reagents at the origin of the mesostructuring during the aerosol process in the presence of reactive functions of the thiol, amine, acidic, and basic types, etc.

### SUMMARY OF THE INVENTION

This invention has as its object a process for cracking tert-alkyl ether(s) selected from among tert-amyl methyl ether (TAME) and ethyl tert-amyl ether (ETAE) for the production of tertiary olefins comprising bringing said tert-alkyl ether(s) into contact with at least one catalyst that is formed by at least one mesostructured hybrid organic-inorganic material that consists of at least two spherical elementary particles, whereby each of said spherical particles consists of a mesostructured matrix with a silicon oxide base to which are linked organic groups with acid terminal reactive functions, whereby said groups represent less than 20 mol % of said matrix that is present in each of said spherical elementary particles, which have a maximum diameter of between 50 nm and 200 μm.

According to the invention, the acid terminal reactive functions of the organic groups that are linked to the mesostructured matrix and that each constitute spherical elementary particles of the material that is present in the catalyst that is used for the implementation of the cracking process according to the invention have acidic properties and are preferably selected from among the following functions: sulfonic acid —SO₃H, carboxylic acid —COOH and derivative, hydroxyl-OH, and phosphonic acid PO₃H. Preferably, said acid terminal reactive functions are sulfonic acid functions —SO₃H.

### ADVANTAGE OF THE INVENTION

The mesostructured hybrid organic-inorganic (MHOI) material that is present in the catalyst that is used for the implementation of the cracking process according to the invention simultaneously has the structural, textural, acid-basic and adsorption properties that are suitable for mesostructured inorganic materials based on silicon and the acidity properties that are suitable for functionalized organic molecules that are fundamentally different from these same properties that are expressed by the inorganic matrix. In addition, whereby said mesostructured MHOI consists of spherical elementary particles having a diameter of controlled size and whereby the diameter of these particles advantageously varies from 50 nm to 200 μm, preferably from 50 nm to 10 μm, preferably from 50 to 300 nm, and even more preferably from 50 to 100 nm, the limited size of these particles as well as their homogeneous shape (spheres) makes it possible to have a better diffusion of reagents and target products of the reaction for cracking tert-alkyl ether(s) according to the process of the invention compared to known MHOI materials from the prior art coming in the form of elementary particles of non-homogeneous shape, i.e., irregular, and with a size that is generally greater than 500 nm. Furthermore, the preparation of said mesostructured MHOI material, which comprises the incorporation of the precursor(s) of the organic groups within the initial solution comprising all of the reagents for the preparation of said mesostructured MHOI, makes it possible to process mesostructured hybrid organic-inorganic materials having organic groups that are preferably located on the walls of pores of the mesostructured matrix that is present in each of the spherical elementary particles of the mesostructured MHOI that is present in the catalyst that is used for the implementation of the cracking process according to the invention. In addition, relative to the mesostructured material syntheses that are known to one skilled in the art, the production of the mesostructured hybrid organic-inorganic material is carried out continuously, the preparation period is reduced (several hours versus 12 to 24 hours by using autoclaving), and the stoichiometry of the non-volatile radicals that are present in the initial solution of the reagents is maintained within the material of the invention.

Surprisingly enough, a catalyst that is formed by such a mesostructured hybrid organic-inorganic material, when it is implemented in a process for cracking tert-alkyl ether(s) selected from among tert-amyl methyl ether (TAME) and ethyl tert-amyl ether (ETAE), leads to improved catalytic performance levels in terms of activity and selectivity toward the desired products, namely the tertiary olefins of the formula 2-methylbut-1-ene and 2-methylbut-2-ene, relative to the performance levels that are obtained by means of a catalyst that is formed by a hybrid organic-inorganic material that is known from the prior art. The yield in target products, which are tertiary olefins (isoamylenes), is thus significantly improved.

### Characterization Technique

The mesostructured MHOI that is present in the catalyst that is used for the implementation of the cracking process according to the invention can be characterized by several analysis techniques and in particular by low-angle x-ray diffraction (low-angle XRD), by nitrogen volumetric analysis (BET), by transmission electron microscopy (TEM), and by HF-induced plasma emission spectrometry (ICP). The presence of the organic groups, and in particular acid terminal reactive functions, can be verified by additional analyses: ¹³C solid nuclear magnetic resonance (¹³C NMR-MAR), acid-basic metering.

The low-angle x-ray diffraction technique (values of the angle 2θ between 0.5° and 6°) makes it possible to characterize the periodicity on the nanometric scale that is generated by the organized mesoporosity of the mesostructured matrix that is present in each of said spherical particles constituting the mesostructured hybrid organic-inorganic material that is present in the catalyst used for the implementation of the cracking process according to the invention. The x-ray diffraction analysis is carried out on powder with a diffractometer that operates by reflection and is equipped with a rear monochromator by using the radiation of copper (wavelength of 1.5406 Å). The peaks that are usually observed in the diffractograms that correspond to a given value of the angle 2θ are associated with inter-reticular distances d₍ₕₖₗ₎ that are characteristic of the structural symmetry of the material, (hkl) being the Miller indices of the reciprocal network, by Bragg's equation: 2 d₍ₕₖₗ₎*sin(θ)=η*λ. This indexing then makes it possible to determine the mesh parameters (abc) of the direct network, whereby the value of these parameters is based on the hexagonal, cubic, cholesteric, lamellar, bicontinuous or vermicular structure that is obtained and is characteristic of the periodic organization of mesopores of said mesostructured hybrid organic-inorganic material.

The nitrogen volumetric analysis that corresponds to the physical adsorption of nitrogen molecules in the porosity of the mesostructured hybrid organic-inorganic (MHOI) material via a gradual increase in pressure at constant temperature gives information on the special textural characteristics (diameter of pores, type of porosity, specific surface area) of the mesostructured MHOI present in the catalyst that is used for the implementation of the cracking process according to the invention. In particular, it makes it possible to access the specific surface area and the mesoporous distribution of said mesostructured material. Specific surface area is defined as the B.E.T. specific surface area (S_{BET} in m²/g) that is determined by nitrogen adsorption in accordance with the ASTM D 3663-78 standard that is established from the BRUNAUER-EMMETT-TELLER method described in the periodical “The Journal of American Society,” 1938, 60, 309. The pore distribution that is representative of a mesopore population centered in a range of 1.5 to 50 nm is determined by the Barrett-Joyner-Halenda (BJH) model. The nitrogen adsorption-desorption isotherm according to the thus obtained BJH model is described in the periodical “The Journal of American Society,” 1951, 73, 373, written by E. P. Barrett, L. G. Joyner and P. P. Halenda. In the disclosure that follows, the diameter of the mesopores φ of the given mesostructured matrix corresponds to the average diameter with the defined nitrogen adsorption as being a diameter such that all of the pores that are smaller than this diameter constitute 50% of the pore volume (Vp) that is measured on the adsorption branch of the nitrogen isotherm. In addition, the form of the nitrogen adsorption isotherm and the hysteresis loop can provide information on the nature of the mesoporosity and on the optional presence of microporosity in the mesostructured matrix of the mesostructured MHOI that is present in the catalyst that is used for the implementation of the cracking process according to the invention.

Regarding the mesostructured MHOI, the difference between the value of the diameter of the pores φ and the mesh parameter a defined by low-angle XRD as described above makes it possible to access the value e where e=a−φ and is characteristic of the thickness of the amorphous walls of the mesostructured matrix that is present in each of the spherical particles constituting said MHOI material that is present in the catalyst that is used for the implementation of the process according to the invention. Said mesh parameter a is connected to the distance d for correlation between pores by a geometric factor that is characteristic of the geometry of the phase. For example, in the case of a hexagonal mesh e=a−φ with a=2*d/\sqrt{3}, in the case of a vermicular structure e=d−φ.

The analysis by transmission electron microscopy (TEM) is a technique that is also widely used to characterize the structure of these materials. The latter makes possible the formation of an image of the solid that is being studied, whereby the contrasts that are observed are characteristic of the structural organization, the texture, or else the morphology of the particles that are observed. The resolution of the technique reaches at most 0.2 nm. In the disclosure that follows, the TEM photos are produced from microtomic sections of the sample so as to visualize a section of a spherical elementary particle of the mesostructured MHOI material that is present in the catalyst that is used for the implementation of the cracking process according to the invention. The analysis of the image also makes it possible to access the parameters d, φ and e that are characteristic of the mesostructured hybrid matrix defined above.

The analysis by ¹³C solid nuclear magnetic resonance (¹³C NMR-MAR) is a technique of choice for characterizing the presence and the nature of organic groups with acid terminal reactive functions that are linked to the mesostructured matrix that is present in each of the spherical particles of the mesostructured MHOI material that is present in the catalyst that is used for the implementation of the cracking process according to the invention. Actually, this technique makes it possible to know the environment that is close to a nucleus that is being considered (short-distance order). It is based on the interaction of atomic nuclei that have a non-zero magnetic moment θ with an external magnetic field B_{O}. This interaction generates, by Zeeman effect, energy levels between which transitions can occur following the application of a radiofrequency-type wave. Each transition frequency corresponds to a nucleus in a given chemical environment. A transition frequency, itself associated with a chemical shift expressed in ppm, is therefore associated with each nucleus. The various NMR spectra of the ¹³C solid have been recorded by means of high-resolution BRUKER Avance 300 and Avance 400 spectrometers. In the case of the study of solids, the anisotropy of chemical shift and the existence of dipolar- or quadripolar-type interactions lead to a great expansion of signals from the spectra obtained. This expansion can be reduced by rapid rotation of the sample along an axis that is inclined by an angle of φ=54° 44′ relative to the direction of the magnetic field B_{O}. Magnetic angle rotation (MAR) is mentioned. In the case of this invention, the chemical shifts of the carbon atoms make it possible to characterize the organic groups. In particular, the carbon atoms of the organic groups that bear the acid terminal reactive functions have specific chemical shifts that are associated with the nature of these functions, thus making it possible to confirm their presence within the mesostructured MHOI material that is present in the catalyst that is used for the implementation of the cracking process according to the invention. Generally, the spectrum that is obtained during the ¹³C NMR-MAR analysis of an organic group of said mesostructured MHOI material is close to the spectrum that is obtained in liquid phase for the corresponding organic precursor, whereby the signals are expanded by solid matrix analysis. For example, the ¹³C NMR spectrum that is obtained for a mesostructured MHOI that consists of spherical elementary particles comprising a silicic mesostructured matrix to which organic groups with terminal reactive functions of formula —(CH₂)₂—C₆H₄—SO₃H are linked and that is obtained by using cetyltrimethylammonium bromide quaternary ammonium salt CH₃(CH₂)₁₅N(CH₃)₃Br (CTAB) as a surfactant, is characteristic of the liquid ¹³C NMR spectrum of the precursor (OMe)₃Si—(CH₂)₂—C₆H₄—SO₃H, whereby the signals are expanded.

When the desired terminal reactive function F is a sulfonic acid function, the characterization of the acidity that is expressed in mmol equivalent of H⁺/g of catalyst (also referred to as “proton exchange capacity”) is carried out by a potentiometric metering via a base, whereby this base is generally sodium hydroxide NaOH or potassium hydroxide KOH.

The morphology and the size distribution of elementary particles have been established by analysis of photos obtained by SEM [scanning electronic microscopy].

### DETAILED DISCLOSURE OF THE INVENTION

This invention has as its object a process for cracking tert-alkyl ether(s) selected from among tert-amyl methyl ether (TAME) and ethyl tert-amyl ether (ETAE) for the production of tertiary olefin(s) comprising bringing said tert-alkyl ether(s) into contact with at least one catalyst that is formed by at least one mesostructured hybrid organic-inorganic material that consists of at least two spherical elementary particles, whereby each of said spherical particles consists of a mesostructured matrix based on silicon oxide to which are linked organic groups with acid terminal reactive functions, whereby said groups represent less than 20 mol % of said matrix present in each of said spherical elementary particles, which have a maximum diameter of between 50 nm and 200 μm.

It is recalled, in order to understand the invention, that the tert-amyl methyl ether (TAME) corresponds to the compound of formula 1,1-dimethylpropyl methyl ether (CH₃—CH₂—C(CH₃)₂—O—CH₃) and that the ethyl tert-amyl ether (ETAE) corresponds to the compound of formula 1,1-dimethylpropyl ethyl ether (CH₃—CH₂—C(CH₃)₂—O—CH₂—CH₃).

In accordance with the process according to the invention, the cracking of TAME leads to the majority production of isoamylenes and methanol and the cracking of ETAE leads to the majority production of isoamylenes and ethanol. The isoamylenes that are produced are 2-methylbut-1-ene and 2-methylbut-2-ene. It involves tertiary olefins that are desired to be produced selectively.

Mesostructured hybrid organic-inorganic (MHOI) material is defined in terms of this invention as a hybrid organic-inorganic material that has an organized porosity on the scale of the mesopores of each of said spherical particles, i.e., an organized porosity on the scale of pores that have a uniform diameter of between 1.5 and 30 nm and preferably between 1.5 and 10 nm and are distributed homogeneously and uniformly in each of said particles constituting the mesostructured hybrid organic-inorganic material (mesostructuring of MHOI) that is present in the catalyst that is used for the implementation of the cracking process according to the invention. It should be noted that a porosity of microporous nature can also result in the overlapping of the surfactant, used during the preparation of the MHOI material that is present in the catalyst that is used in the process according to the invention, with the inorganic wall at the organic-inorganic interface that is developed during the mesostructuring of the inorganic component of said material.

In accordance with the invention, the silicon oxide-based matrix, encompassed in each of said spherical elementary particles constituting said mesostructured MHOI, is mesostructured: it has mesopores that have a uniform diameter, i.e., identical for each mesopore, of between 1.5 and 30 nm and preferably between 1.5 and 10 nm, distributed homogeneously and uniformly in each of said spherical particles. The material that is located between the mesopores of each of said spherical particles is amorphous and forms walls, or panels, whose thickness is between 1 and 20 nm. The thickness of the walls corresponds to the mean distance that separates a first pore from a second pore, whereby the second pore is the pore that is the closest to said first pore. The organization of the mesoporosity that is described above leads to a structuring of the silicon oxide-based matrix, which can be hexagonal, cubic, cholesteric, lamellar, bicontinuous or vermicular.

According to the invention, the acid terminal reactive functions of the organic groups that are linked to the mesostructured matrix and that each constitute spherical elementary particles of the material that is present in the catalyst that is used for the implementation of the cracking process according to the invention have acidity properties and are preferably selected from among the following functions: sulfonic acid —SO₃H, carboxylic acid —COOH and derivative, hydroxyl-OH, and phosphonic acid PO₃H. Preferably, said acid terminal reactive functions are sulfonic acid functions —SO₃H. Said organic groups are linked to the mesostructured matrix by covalent bonds. They represent less than 20 mol % of said matrix that is present in each of said spherical elementary particles, and preferably represent from 0.1 to 19.5 mol %, and very preferably 0.5 to 18 mol % of said matrix that is present in each of said spherical elementary particles. Said organic groups that are linked to said mesostructured matrix and the acid terminal reactive functions that they carry at their ends can be identical and can be obtained from the use of a single organosilane precursor as described below in this description or can be different and obtained from the use of at least two different organosilane precursors, with the proviso that the different acid terminal reactive functions that are considered are compatible with the process, i.e., that they do not react with one another and do not cause the precipitation of the precursors in the initial solution that is used for the preparation of the mesostructured MHOI as described below in this description.

According to a particular embodiment of the mesostructured hybrid organic-inorganic material that is present in the catalyst that is used for the implementation of the process according to the invention, the silicon oxide-based mesostructured matrix that is present in each of said spherical particles of said material is entirely silicic.

According to another particular embodiment of the mesostructured hybrid organic-inorganic material that is present in the catalyst that is used for the implementation of the process according to the invention, the silicon oxide-based mesostructured matrix that is present in each of said spherical particles of said material comprises at least one element Z that is selected from the group that consists of aluminum, titanium, tungsten, zirconium and cerium. Preferably, the element Z is aluminum.

The spherical elementary particles that constitute the mesostructured MHOI that is present in the catalyst that is used for the implementation for the process according to the invention have a diameter that is advantageously between 50 nm and 200 μm, preferably between 50 nm and 10 μm, more preferably between 50 and 300 nm, and even more preferably between 50 and 100 nm. More specifically, they are present in said mesostructured MHOI material that forms the catalyst that is used for the implementation of the process according to the invention in the form of aggregates.

The mesostructured MHOI that is present in the catalyst that is used for the implementation of the cracking process according to the invention advantageously has a specific surface area of between 100 and 1500 m²/g, and very advantageously between 300 and 1000 m²/g.

The catalyst that is used for the implementation of the cracking process according to the invention advantageously consists integrally of said mesostructured hybrid organic-inorganic material.

Said mesostructured hybrid organic-inorganic (MHOI) material as described above and present in the catalyst that is used for the implementation of the cracking process according to the invention is disclosed in particular in the patent application FR-A-2,894,580.

Said mesostructured MHOI that is present in the catalyst that is used for the implementation of the cracking process according to the invention can be obtained according to two preparation processes.

A first process for preparation of the mesostructured MHOI that is present in the catalyst that is used for the implementation of the process according to the invention comprises:

a) The mixing in solution of at least one surfactant, at least one silicic precursor, optionally at least one precursor of at least one element Z that is selected from the group that consists of aluminum, titanium, tungsten, zirconium and cerium and at least one organosilane precursor that has at least one acid terminal reactive function selected from among the following functions: sulfonic acid —SO₃H, carboxylic acid —COOH and derivative, hydroxyl-OH and phosphonic acid PO₃H;

b) The atomization by aerosol of said solution that is obtained in stage a) to lead to the formation of spherical droplets with a diameter of less than 300 μm;

c) The drying of said droplets;

d) The elimination of said surfactant for obtaining a MHOI with organized and uniform porosity.

According to stage a) of said first process for preparation of the mesostructured MHOI that is present in the catalyst that is used in the process according to the invention, the silicic precursor and optionally the precursor of at least one element Z are inorganic oxide precursors that are well known to one skilled in the art. The silicic precursor is obtained from an organometallic precursor of formula Si(OR₁)₄ where R₁═H, methyl, ethyl. The precursor of the element Z can be any organometallic compound that comprises the element Z of formula Z(OR₂)ₙ with, for example, R₂=methyl, ethyl, isopropyl, n-butyl, s-butyl or t-butyl, etc. The precursor of the element Z can also be an oxide, a metallic hydroxide or a metallic chloride of formula Z(Cl)ₙ.

Said organic groups are introduced within the mesostructured MHOI that is present in the catalyst that is used for the implementation of the process according to the invention by using organosilane precursors in accordance with stage a) of the first process for preparation of the mesostructured MHOI. Any organoalkoxysilane or organochlorosilane having one or more acid terminal reactive functions can be used. In particular, an organoalkoxysilane of dendritic nature can be used, whereby the latter is a monodisperse hyperbranched polymer of nanoscopic size that consists of a reactive nucleus that is generally alkoxysilane and that has a large number of reactive terminal functions on its periphery.

Preferably, the organoalkoxysilane and organochlorosilane precursors are respectively characterized by the following general formulas: (OR)₄₋ₓSi—(R′—F)ₓ and (Cl)₄₋ₓSi—(R′—F)ₓ (x=1 or 2) with R═H, methyl, ethyl, R′=alkyl chains, phenylalkyl chains, and arylalkyl chains, and whereby F is a acid terminal reactive function. The fragment alkoxysilane —Si(OR′)₄₋ₓ(x=1 or 2) or chlorosilane —Si(Cl)₄₋ₓ(x=1 or 2) of the possible precursor makes it possible, via the hydrolysis-condensation reactions, to incorporate the organic group(s) R—F into the inorganic framework via the covalent bond of the silicon with the fragment(s) —R— of the organic group (generally an Si—C bond). The fragment(s) —R— of the organic group can be considered as a spacer between the inorganic framework and the acid terminal reactive function that is being considered. The acid terminal reactive function F is selected from among the following functions: sulfonic acid —SO₃H, carboxylic acid —COOH and derivative, hydroxyl-OH, and phosphonic acid PO₃H. Preferably, the terminal reactive functions that are being considered are the functions —SO₃H. In the preferred case where the desired acid terminal reactive function F is a sulfonic acid function, a usable organoalkoxysilane precursor is in particular the precursor (chlorosulfonylphenylethyl acid)trimethoxysilane (OMe)₃Si—(CH₂)₂—C₆H₄—SO₂Cl, and a usable organochlorosilane precursor is in particular the precursor (chlorosulfonylphenylethyl acid) trichlorosilane (Cl)₃Si—(CH₂)₂—C₆H₄—SO₂Cl.

The surfactant that is used for the preparation of the mixture according to stage a) of the first process for preparation of the mesostructured MHOI that is present in the catalyst that is used for the implementation of the cracking process according to the invention is an ionic or non-ionic surfactant, or a mixture of the two. Preferably, the ionic surfactant is selected from among the phosphonium and ammonium ions, and very preferably from among the quaternary ammonium salts such as cetyltrimethylammonium bromide (CTAB). Preferably, the non-ionic surfactant can be any copolymer that has at least two parts of different polarities that impart amphiphilic macromolecule properties. These copolymers can be part of the non-exhaustive list of the following copolymer families: the fluorinated copolymers (—[CH₂—CH₂—CH₂—CH₂—O—CO—R2— with R2=C₄F₉, C₈F₁₇, etc.), the biological copolymers such as the amino polyacids (polylysine, alginates, etc.), the dendrimers, the block copolymers that consist of poly(alkylene oxide) chains, and any other copolymer with an amphiphilic nature that is known to one skilled in the art (S. Förster, M. Antionnetti, Adv. Mater, 1998, 10, 195-217; S. Forster, T. Plantenberg, Angew. Chem. Int. Ed, 2002, 41, 688-714; H. Cölfen, Macromol. Rapid Commun, 2001, 22, 219-252). Preferably, within the scope of this invention, a block copolymer that consists of poly(alkylene oxide) chains is used. Said block copolymer is preferably a block copolymer that has two, three or four blocks, whereby each block consists of a poly(alkylene oxide) chain. For a two-block copolymer, one of the blocks consists of a poly(alkylene oxide) chain that is hydrophilic in nature and the other block consists of a poly(alkylene oxide) chain that is hydrophobic in nature. For a three-block copolymer, two of the blocks consist of a poly(alkylene oxide) chain that is hydrophilic in nature while the other block, located between the two blocks with hydrophilic parts, consists of a poly(alkylene oxide) chain that is hydrophobic in nature. Preferably, in the case of a three-block copolymer, the poly(alkylene oxide) chains that are hydrophilic in nature are poly(ethylene oxide) chains that are denoted as (PEO)ₓ, and (PEO)_{z}, and the poly(alkylene oxide) chains that are hydrophobic in nature are poly(propylene oxide) chains that are denoted (PPO)_{y}, poly(butylene oxide) chains, or mixed chains of which each chain is a mixture of several alkylene oxide monomers. Very preferably, in the case of a three-block copolymer, a compound of formula (PEO)ₙ—(PPO)_{y}—(PEO)_{z} is used, where x is between 5 and 300, and y is between 33 and 300, and z is between 5 and 300. Preferably, the values of x and z are identical. Very advantageously, a compound in which x=20, y=70, and z=20 (P123) and a compound in which x=106, y=70, and z=106 (F127) are used. The commercial non-ionic surfactants that are known under the names of Pluronic (BASF), Tetronic (BASF), Triton (Sigma), Tergitol (Union Carbide), and Brij (Aldrich) can be used as non-ionic surfactants in stage a) of the process for preparation of said mesostructured MHOI that is present in the catalyst that is used for the implementation of the cracking process according to the invention. For a four-block copolymer, two of the blocks consist of a poly(alkylene oxide) chain that is hydrophilic in nature, and the other two blocks consist of a poly(alkylene oxide) chain that is hydrophobic in nature.

The atomization stage of the mixture according to stage b) of said first process for preparation of the mesostructured MHOI that is present in the catalyst that is used for the implementation of the cracking process according to the invention produces spherical droplets with a diameter that is less than or equal to 300 μm, and preferably in a range of between 50 nm and 30 μm. The size distribution of these droplets is lognormal. The aerosol generator that is used here is a commercial device of model 9306 that is provided by TSI, having a 6-jet atomizer. The atomization of the solution is done in a chamber into which are sent a carrier gas, an O₂/N₂ mixture (dry air), under a pressure P that is equal to about 1 bar (1 bar=10⁵ pascal).

In accordance with stage c) of said first process for preparation of the mesostructured MHOI that is present in the catalyst that is used for the implementation of the cracking process according to the invention, drying of said droplets produced during said stage b) is initiated. This drying is carried out by the transport of said droplets via the carrying gas, the O₂/N₂ mixture, in glass tubes, which leads to the gradual evaporation of the solution, for example of the acidic aqueous organic solution as specified in the remainder of this disclosure, and thus to obtaining spherical elementary particles. This drying is again improved by a passage of said particles into a furnace whose temperature can be adjusted, whereby the usual temperature range varies from 50° C. to 600° C., and preferably from 80° C. to 400° C. The dwell time of the particles in the furnace is on the order of a second. The particles are then recovered in a filter and constitute the mesostructured MHOI that is present in the catalyst that is used in the cracking process according to the invention. A pump that is placed at the end of the circuit promotes the channeling of radicals into the experimental aerosol device. The drying of the droplets according to stage c) of said first process for preparation of the mesostructured MHOI that is present in the catalyst that is used in the cracking process according to the invention is advantageously followed by a passage into the oven at a temperature of between 50 and 150° C.

The elimination of the surfactant during stage d) of said first process for preparation of the mesostructured MHOI that is present in the catalyst that is used in the process according to the invention is advantageously carried out by processes of chemical extraction or via suitable heat treatments so as to selectively decompose the organic surfactant without modifying the organic groups of the mesostructured MHOI that is present in said catalyst. Preferably, the surfactant is eliminated by reflux washing in an organic solvent such as ethanol.

A possible variant to said first process for preparation of the mesostructured MHOI that is present in the catalyst that is used in the cracking process according to the invention consists in deferring by 1 to 4 hours the addition of at least one organosilane precursor that has at least one acid terminal reactive function that is selected from among the following functions: sulfonic acid —SO₃H, carboxylic acid —COOH and derivative, hydroxyl-OH and phosphonic acid PO₃H relative to the other reagents that are used for the implementation of said stage a) of said first process for preparation of the mesostructured MHOI that is present in said catalyst that is used for the implementation of the cracking process according to the invention.

In a second process for preparation of the mesostructured MHOI that is present in the catalyst that is used for the implementation of the cracking process according to the invention that is called “second process for preparation of the mesostructured MHOI that is present in the catalyst that is used for the implementation of the cracking process according to the invention” below, the precursors of the organic groups that are introduced into the initial solution of the reagents have intermediate organic groups, and the desired acid terminal reactive functions will be obtained only after a chemical treatment of these intermediate groups. More specifically, said second process for preparation of the mesostructured MHOI that is present in the catalyst that is used for the implementation of the cracking process according to the invention comprises:

a′) The mixing in solution of at least one surfactant, at least one silicic precursor, optionally at least one precursor of at least one element Z that is selected from the group that consists of aluminum, titanium, tungsten, zirconium and cerium, and at least one organosilane precursor that has at least one intermediate organic group,

b′) The atomization by aerosol of said solution that is obtained in stage a′) for leading to the formation of spherical droplets with a diameter that is less than 300 μm,

c′) The drying of said droplets,

d′) The elimination of said surfactant for obtaining an MHOI material with organized and uniform porosity;

e′) The transformation of the intermediate organic group of the MHOI material that is obtained in stage d′) into an organic group that has the acid terminal reactive function that is desired by suitable chemical treatments.

In accordance with stage a′) of said second process for preparation of the mesostructured MHOI that is present in the catalyst that is used for the implementation of the cracking process according to the invention, the silicic precursor, optionally the precursor of at least one element Z that is selected from among aluminum, titanium, tungsten, zirconium and cerium, and the surfactant that is used for the preparation of the mixture according to said stage a′) are identical to those that are defined for stage a) of said first process for preparation of the mesostructured MHOI that is present in the catalyst that is used for the implementation of the cracking process according to the invention. The intermediate organic groups are introduced into the solution of said stage a′) via the use of organosilane precursors such as those described for said stage a) of said first process for preparation of the mesostructured MHOI that is present in the catalyst that is used for the implementation of the cracking process according to the invention. Said intermediate organic groups are carefully selected so as to lead—after chemical treatments—to the formation of organic groups —R—F where F is the desired acid terminal reactive function that is selected from among the following functions: sulfonic acid —SO₃H, carboxylic acid —COOH and derivative, hydroxyl-OH, and phosphonic acid PO₃H. Preferably, the reactive terminal functions that are being considered are the sulfonic acid functions —SO₃H. For example, when the desired terminal reactive function F is a sulfonic acid function, the intermediate organic group can have a thiol function or be a phenylalkyl chain that could respectively undergo an oxidation stage or a sulfonation stage to lead to the desired function —SO₃H.

The stages b′), c′), and d′) of said second process for preparation of the mesostructured MHOI that is present in the catalyst that is used for the implementation of the cracking process according to the invention are in all respects similar to stages b), c) and d) of said first process for preparation of the mesostructured MHOI that is present in the catalyst that is used for the implementation of the cracking process according to the invention.

The chemical treatments that lead to the transformation of the intermediate organic group into an organic group that has the desired acid terminal reactive function according to stage e′) of said second process for preparation are selected so as not to damage the mesostructuring of the hybrid material MHOI that is obtained in stage d′) and to preserve as well as possible the content of organic groups that are introduced into the initial solution of stage a′). In the preferred case where the desired terminal reactive function is the sulfonic acid function, the intermediate organic group can have a thiol function or be a phenylalkyl chain. Concerning an intermediate organic group that has a thiol function, the latter is advantageously oxidized according to the standard processes that are known to one skilled in the art, such as treatments with hydrogen peroxide, with nitric acid, with barium permanganate, etc. After oxidation, the material that is obtained is washed with water and oven-dried at a temperature of between 50° C. and 150° C. Concerning a phenylalkyl organic intermediate group, the sulfonation of the aromatic cycle is initiated, which is carried out according to conventional methods that are known to one skilled in the art: treatments with chlorosulfonic acid, with concentrated sulfuric acid, with sulfur oxide SO₃, etc.

A first possible variant to said second process for preparation of the mesostructured MHOI that is present in the catalyst that is used in the cracking process according to the invention consists in carrying out stage e′) and stage a′) simultaneously.

A second possible variant to said second process for preparation of the mesostructured MHOI that is present in the catalyst that is used in the cracking process according to the invention consists in deferring by 1 to 4 hours the addition of at least one organosilane precursor that has at least one intermediate organic group relative to the other reagents that are used for the implementation of said stage a′) of said second process for preparation of the mesostructured MHOI that is present in the catalyst that is used for the implementation of the cracking process according to the invention.

The solution into which are mixed all of the reagents according to the stages a) and a′) respectively of the first and the second processes for preparation of the mesostructured MHOI that constitutes the catalyst that is prepared according to the invention can be acidic, neutral or basic. Preferably, said solution is acidic and has a maximum pH that is equal to 3, preferably between 0 and 2. The acids that are used to obtain an acid solution with a maximum pH that is equal to 3 are, in a non-exhaustive way, hydrochloric acid, sulfuric acid, and nitric acid. Said solution can be aqueous or can be a water-organic solvent mixture, whereby the organic solvent is preferably a water-miscible polar solvent, in particular THF or an alcohol, preferably ethanol.

Said solution can also be practically organic, preferably practically alcoholic, whereby the amount of water is such that the hydrolysis of the inorganic precursors and organosilanes is ensured stoichiometrically. Very preferably, said solution consists of acidic aqueous organic mixtures and very preferably of acid water-alcohol mixtures. This latter characteristic is valid for the two processes for preparation of the mesostructured MHOI described above.

The initial concentration of surfactant that is introduced into the mixture according to the stages a) and a′) of the first and the second processes for preparation of the mesostructured MHOI that is present in the catalyst that is used for the implementation of the cracking process according to the invention is defined by cₒ, and cₒ is defined relative to the critical micellar concentration (Cmc) that is well known to one skilled in the art. The Cmc is the boundary concentration beyond which the self-assembly phenomenon of the surfactant molecules occurs in the solution. The concentration cₒ can be less than, equal to, or greater than the Cmc; preferably it is less than the Cmc. In one preferred implementation of each of the two processes for preparation of the mesostructured MHOI that is described above, the concentration cₒ is less than the Cmc and said solution that is targeted at each of stages a) and a′) of each of the two processes for preparation of the mesostructured MHOI described above is an acid water-alcohol mixture.

In the case where the solution that is targeted at each of the stages a) and a′) of each of the two processes for preparation of mesostructured MHOI that is present in the catalyst that is used for the implementation of the cracking process according to the invention is a water-organic solvent mixture, preferably acidic, it is preferred during each of said stages a) and a′) that the surfactant concentration at the origin of the mesostructuring of the matrix that is present be less than the critical micellar concentration, such that the evaporation of said preferably acidic aqueous organic solution, during each of stages b) and b′) by the aerosol technique, causes a phenomenon of micellization or self-assembly leading to the mesostructuring of the mesostructured MHOI matrix. When cₒ<Cmc, the mesostructuring of the matrix that is present in each of the spherical particles that constitute the mesostructured MHOI, prepared according to one of the two mesostructured MHOI processes described above in this description, is the result of a gradual concentration, within each droplet, of surfactant, silicic precursor, organosilane precursor, and optionally precursor with at least one element Z, up to a concentration of surfactant c>Cmc resulting from an evaporation of the preferably acidic aqueous organic solution.

In general, the increase of the combined concentration of the silicic precursor, the organosilane precursor, optionally the precursor of at least one element Z and the surfactant causes the precipitation of the hydrolyzed silicic precursor, the hydrolyzed organosilane precursor, and optionally the precursor that is hydrolyzed with at least one element Z around the self-organized surfactant. The structuring of the mesostructured MHOI results therefrom.

The following interactions: inorganic/inorganic phases, organic/organic phases, and organic/inorganic phases lead to a self-assembly mechanism that works with the condensation of the hydrolyzed silicic precursor, the hydrolyzed organosilane precursor, and optionally the precursor that is hydrolyzed by at least one element Z around the self-organized surfactant. More specifically, relating to the behavior in solution of the organosilane precursor during self-assembly phenomena caused by evaporation, the reactions of hydrolysis—condensation of the alkoxysilane or chlorosilane fragment will make possible the hooking of the organic group into the inorganic matrix by reaction with the hydrolyzed silicic precursor, and optionally the hydrolyzed precursor by at least one element Z, while the organic group, by affinity with the organic surfactant, will have a tendency to be located in the micellar phase that is defined by the surfactant. This dual compatibility of the organosilane precursor that is hydrolyzed for the inorganic phase under construction, on the one hand, and for the organic phase that is combined with surfactant, on the other hand, is at the origin of the preferred location of the organic groups and therefore acidic terminal reactive functions that are present in the final material at the walls of the pores of the mesostructure.

The aerosol technique is particularly advantageous for the implementation of the stages b) and b′) of each of the two processes for preparation of the mesostructured MHOI that is present in the catalyst that is used for the implementation of the cracking process according to the invention so as to force the reagents that are present in the initial solution to interact with one another, whereby no loss of material except for the solvents is possible. All of the silicon elements, organic groups and optionally Z elements that are present initially are thus perfectly preserved throughout each of the two processes for preparation of the mesostructured MHOI that is present in the catalyst that is used for the implementation of the cracking process according to the invention while these reagents are partially eliminated during stages of filtration and washing encountered in conventional synthesis processes that are known to one skilled in the art.

The catalyst that is used for the implementation of the cracking process according to the invention can be obtained in the form of powder, balls, pellets, granules, or extrudates, whereby the shaping operations are carried out by the conventional techniques that are known to one skilled in the art. Preferably, said catalyst is obtained in the form of powder, which consists of agglomerates of particles that have a diameter of between 500 μm and 1.5 mm, which facilitates the diffusion of the tert-alkyl ether(s) into the framework of the implementation of the cracking process according to the invention. A diameter that is centered on 1 mm is the ideal compromise between a moderate pressure drop in the reactor and an optimum radial diffusion of reagents.

The process for cracking tert-alkyl ether(s) selected from among the tert-amyl methyl ether (TAME) and the ethyl tert-amyl ether (ETAE) according to the invention is generally implemented in at least one reaction zone that comprises at least one reactor, whereby each reactor operates either in a fixed bed, or a moving bed, or an expanded bed or else a fluidized bed. It is possible to combine the various modes of operation of the reactor. In addition, the reaction zone reactor(s) can operate, independently of one another in the case of the presence of at least two reactors, in upward flow or in downward flow. It is possible to combine the two circulation modes when said zone comprises at least two reactors, i.e., when at least one reactor operates in upward flow and at least one reactor operates in downward flow. It is also possible to use at least one radial-type reactor.

The cracking process according to the invention is advantageously applied to a hydrocarbon feedstock that comprises at least 90% by weight of at least one tert-alkyl ether that is selected from among tert-amyl methyl ether (TAME) and ethyl tert-amyl ether (ETAE). The remainder of the feedstock advantageously comprises hydrocarbons that may or may not be saturated. It involves in particular olefins, naphthenes and paraffins.

The cracking process according to the invention is advantageously implemented under the following operating conditions: the temperature is between 100 and 200° C., preferably between 120 and 180° C., the pressure is between 5 and 10.10⁵ Pa, and the VVH (hourly volume of feedstock related to the volume of catalyst) is between 4 and 40 h⁻¹.

In contrast, the reaction for cracking or decomposition of the ethers is highly endothermic. It can therefore give rise to significant temperature gradients in the reactor, which generally involves two major drawbacks: 1) a portion of the catalyst does not operate under optimum heat conditions. Actually, too low a temperature limits the catalytic activity, both from a kinetic standpoint and from a thermodynamic standpoint, and 2) there is a selectivity gradient of the reaction that may be difficult to monitor. So as to limit the impact of endothermicity of the reaction, it is proposed to perform the process of the invention according to various preferred implementations.

One of the preferred implementations of the process according to the invention is such that any reaction zone comprises at least one reactor in a fixed bed, operating in upward flow or in downward flow, and that said reactor is preferably equipped with means that make it possible to provide calories to various locations inside the reactor. By way of non-restrictive illustration, it is possible to cite the example of the multitubular reactor, as described on page 1311 of the work “Le Pétrole, Raffinage et G6nie Chimique [Petroleum, Refining, and Chemical Engineering],” Volume II, by Pierre Wuithier (TECHNIP Editions). One of the advantages of such an implementation is that the supply of calories to part or all of the length of the reactor makes it possible to homogenize the temperature, at least in part, and thus to recover the endothermic phenomenon. Mention is made in general of a technique that makes it possible to work as much as possible in the “near isotherm.”

Another of the preferred implementations of the process according to the invention is such that at least one, preferably the entire reaction zone, comprises at least two reactors that are arranged in series and are equipped with at least one intermediate heat exchange means so as to supply calories to the inlet of at least one reactor, preferably each reactor, and optionally also to the inside of at least one, preferably each reactor (as indicated according to the preceding preferred implementation).

Another of the preferred implementations of the process according to the invention, independently or not of the preceding implementation, is such that any reaction zone comprises at least one reactor that is selected from among the reactors that operate in a moving bed, in an expanded bed, or in a fluidized bed. One of the advantages of such an implementation is that said reactor improves heat exchanges at least in part and thus goes in the direction of a homogenization of the temperature (i.e., a reduction of the temperature gradient and therefore an optimization of the operation of the catalyst). A first variant of such an implementation is such that said reactor comprises at least one recirculation means (around the reactor(s) concerned). A second variant of such an implementation is such that the geometric shape is suitable, i.e., such that the linear speed can be significant within the reactor; in practice, this is reflected by, for example, a small reactor diameter. A combination of the two variants cited above can also be implemented. One of the advantages of such an implementation with recirculation is a great flexibility with regard to the feedstock to be treated: the dwell time (or the linear speed) in the reaction zone can be kept stable despite variations in flow rate of the feedstock to be treated. Consequently, such an implementation comprises at least two advantages, which are facility of operation of the process and better monitoring of the secondary reactions, due to the total control of the VVH, i.e., an optimization of the yields in tertiary olefins.

Another of the preferred implementations of the process according to the invention, independently or not of the preceding implementations, is such that at least one, preferably the entire reaction zone comprises at least two, preferably 2 to 10, reactors in parallel, preferably having independent heating systems. One of the advantages of such an implementation is that the endothermic phenomenon is then distributed into at least two reactors, which leads to obtaining reactors in which, and for each of them, the temperature gradients are lower. In said implementation, it is preferable, without this being restrictive, to work in a fixed bed in each of the reactors in parallel, in upward or downward circulation. Another of the advantages of such an implementation is a very great flexibility with regard to the feedstock to be treated: according to the quantity of feedstock to be treated, it is possible to supply all or only a part (or a certain number) of the reactors in parallel. The dwell time in the reactor can thus be kept stable despite variation in flow rate of the feedstock to be treated. Consequently, such an implementation comprises at least two advantages, which are a facility of operation of the process and a better monitoring of the secondary reactions, i.e., an optimization of the yields both in tertiary olefins and in alcohol.

Regardless of the implementation of the process according to the invention, the process can comprise at least one recycling of at least one portion of the effluent from the reaction zone in said reaction zone, so as to reintroduce into said zone at least one portion of the ether that has not reacted, after purification (i.e., elimination of the major portion of the products of the reaction, i.e., tertiary olefin(s) and alcohol(s), and possible secondary products, such as dimers).

### EXAMPLES

### Example 1

### Preparation of a Catalyst that is Formed by a Mesostructured Hybrid Organic-Inorganic Material that Consists of a Silicic Mesostructured Matrix to which are Linked Organic Groups —(CH₂)₃—SO₃H with 18 Mol % of the Inorganic Matrix that is Obtained According to the Second Process for Preparation of the Mesostructured MHOI

8 g of tetraethyl orthosilicate (TEOS) and 2.0 g of mercaptopropyl triethoxysilane are added to a solution that contains 65 g of ethanol, 34 g of water, 81 μl of HCl (35% by mass) and 3.08 g of surfactant CTAB. The batch is left to stir at ambient temperature for 2 hours and 30 minutes until the precursors are completely dissolved. The entire mixture is sent into the atomization chamber of the aerosol generator, and the solution is sprayed in the form of fine droplets under the action of the carrier gas (dry air) that is introduced under pressure (P=1 bar) as it was described in the description above. The droplets are dried according to the operating procedure that is described in the disclosure of the invention above. The temperature of the drying furnace is set at 350° C. The recovered powder is then consolidated by running it through the oven at 130° C. for 60 hours. The CTAB surfactant is extracted from the MHOI material by reflux washing with ethanol for 2 hours (100 ml of solvent/g of product). The thus obtained hybrid material is then oxidized by the hydrogen peroxide: 1 g of powder is treated by 37 ml of hydrogen peroxide (H₂O₂) at 30% by mass for 24 hours while being stirred. After oxidation, the powder is washed with water, acidified with 0.05 M sulfuric acid, then again washed copiously with water until the pH is neutral. After a final rinsing with ethanol, the mesostructured hybrid MHOI material is dried in the oven for one night at 60° C. The solid is characterized by low-angle XRD, by nitrogen volumetric analysis by TEM, by ¹³C NMR-MAR, by basic metering with soda and by ICP. The TEM analysis shows that the final hybrid material has an organized mesoporosity that is characterized by a 2D hexagonal structure. The nitrogen volumetric analysis leads to a specific surface area of the final hybrid material of S_{BET}=565 m²/g and to a mesoporous diameter of φ=2.1 nm. The low-angle XRD analysis leads to the visualization of a correlation peak at the angle 2θ=3.0°. Bragg's equation 2d*sin(1.5)=1.5406 makes it possible to calculate the distance d for correlation between the pores of the mesostructured matrix and therefore the mesh parameter a according to the equation a=2*d/\sqrt{3}, or a=3.5 nm. The thickness of the walls of the mesostructured MHOI material defined by e=a−φ is therefore e=1.4 nm. A SEM picture of thus obtained spherical elementary particles indicates that these particles have a size that is characterized by a diameter that varies from 50 to 700 nm, whereby the size distribution of these particles is centered around 300 nm. The experimental molar percentage in organic groups relative to the silicic matrix is 18% according to the ICP data. The mesostructured MHOI material that is obtained comes in the form of powder, which is made into pellets, crushed and then sieved according to a grain size such that the diameter of the agglomerates that are obtained is between 0.8 and 1.2 mm.

The catalyst C1 is thus obtained.

### Example 2 (Invention)

### Catalytic Performance Levels of the Catalyst C1 that is Tested in a Cracking Reaction of Ethyl Tert-Amyl Ether (ETAE)

1.8 g of catalyst C1 is introduced into a 500 cm autoclave reactor. 250 cm of a feedstock that consists of 95% by weight of ethyl tert-amyl ether (ETAE) and 5% by weight of heptane are also introduced into said autoclave reactor.

The cracking reaction of the ethyl tert-amyl ether is carried out at a temperature that is equal to 140° C. under a pressure that is equal to 2·10⁵ Pa and while being stirred at a speed of 200 rpm.

An analysis (by gas phase chromatography) of the composition of the reagents is carried out as soon as the temperature in the autoclave reactor has reached 140° C. This moment is denoted t0. At the end of 4.5 hours, the autoclave reactor is placed in dry ice to stop the reaction, and the liquid effluent is analyzed after cooling.

The catalytic performance levels of the catalyst C1 are determined in terms of the molar conversion of ETAE and selectivity of isoamylenes, namely 2-methylbut-1-ene and 2-methylbut-2-ene that correspond to the tertiary olefins that are sought in the cracking reaction of ethyl tert-amyl ether.

The molar conversion of ETAE is calculated as follows:

$${C}_{ETAE} = 100 \cdot \frac{{N}_{ETAE }^{0} - {N}_{ETAE}^{t}}{{N}_{ETAE}^{0}}$$

N^{O}_{ETAE} is the number of mols of ETAE present in the initial feedstock before its introduction into the autoclave reactor, and N^{t}_{ETAE} is the number of mols of ETAE present in the autoclave reactor at time t=4.5 hours.

The tertiary isoamylene selectivity is calculated as follows:

$${S}_{{C}_{s} T} = \frac{{N}_{2 Me 2 Bu}^{t} + {N}_{2 Me 1 Bu}^{t}}{{N}_{ETAE}^{0} - {N}_{ETAE}^{t}}$$

N^{O}_{ETAE} and N^{t}_{ETAE} are as defined for the calculation of the conversion.

N^{t}_{2Me2Bu} is the number of mols of 2-methylbut-2-ene present in the autoclave reactor at time t=4.5 hours, and N^{t}_{2MelBu} is the number of mols of 2-methylbut-1-ene present in the autoclave reactor at time t=4.5 hours.

The isoamylene yield is defined as being the product of the conversion of ETAE by the tertiary isoamylene selectivity.

The results are presented in Table 1 below.

TABLE 1
Catalytic Performance Levels of the Catalyst C1

Time (Hours)

| | tO | 4.5 |
|---|---|---|
| ETAE Conversion (% mol) | 43.13 | 85.34 |
| Isoamylene Selectivity | 86.17 | 85.12 |
| (% mol) | | |
| Isoamylene Yield (%) | 37.16 | 72.65 |

### Example 3 (For Comparison)

### Catalytic Performance Levels of a Non-Mesostructured Catalyst C2 Tested in a Cracking Reaction of Ethyl Tert-Amyl Ether (ETAE)

For this example, the solid that is known under the commercial reference DELOXAN ASP, marketed by the Degussa Company, is used as a catalyst. It is a polysiloxane-type solid that is grafted by at least one sulfonic acid-type organic group and comes in the form of particles with a diameter of between 0.4 and 1.6 mm. This solid is described in particular in the patents U.S. Pat. No. 5,354,831 and U.S. Pat. No. 5,380,791. This solid is not mesostructured and does not come in the form of spherical elementary particles. It is denoted catalyst C2.

It is used for the implementation of the cracking reaction of the ethyl tert-amyl ether that is carried out under the same operating conditions as those provided in Example 2.

The results of the catalytic performance levels of the catalyst C2 appear in Table 2.

TABLE 2
Catalytic Performance Levels of Catalyst C2

Time (Hours)

| | 0 | 4.5 |
|---|---|---|
| ETAE Conversion (% mol) | 7.71 | 75.47 |
| Isoamylene Selectivity | 90.50 | 82.19 |
| (% mol) | | |
| Yield (%) | 6.98 | 62.03 |

The results that appear in Tables 1 and 2 demonstrate that the conversion of the ETAE is already even better with the catalyst C1 than with the catalyst C2 as soon as the reaction begins: the temperature rise time to reach the reaction temperature of 140° C. is already enough to obtain a conversion of ETAE that is equal to 43.13 mol % with the catalyst C1 versus only a conversion of 7.71 mol % with the catalyst C2. This tendency is observed throughout the reaction. At the end of the reaction, the conversion of the ETAE is equal to 85.34 mol % when the reaction is carried out in the presence of the catalyst C1 versus 75.47 mol % when the reaction is carried out in the presence of the catalyst C2: these results prove that the catalyst C1 is much more active than the catalyst C2. In addition, the catalyst C1 is also more selective than the catalyst C2, as the results of isoamylene selectivity demonstrate, which correspond to tertiary olefins that are produced during the reaction and that are the desired products of the cracking reaction of ETAE. By more strongly promoting the production of the desired tertiary olefins, the catalyst C1 limits the secondary reactions such as the dimerization and hydration of the isoamylenes, the etherification of the secondary olefins such as pent-2-ene (cis and trans isomers), the intermolecular dehydration of the ethanol that is formed, the hydrogen transfer reactions, and the isomerization reactions of the position of the double bond. In contrast, the catalyst C2 promotes said secondary reactions at the expense of the primary reaction desired, which leads to the production of isoamylenes as target products. Thus, a much better isoamylene yield results when the reaction is carried out in the presence of the catalyst C1 relative to the one that is obtained when the reaction is carried out in the presence of the catalyst C2. The improved catalytic performance levels of the catalyst C1 relative to those of the catalyst C2 are linked to the mesostructuring of the silicon oxide-based matrix that is present in each of the elementary particles that constitute the catalyst C1 as well as the sphericity and the monitoring of the diameter of each of said particles. They can also be attributed to a better diffusion of ethyl tert-amyl ether (ETAE) at the acid sites of the catalyst C1 relative to those of catalyst C2 whereas C1 comprises fewer acid functions relative to the catalyst C2 (0.7 mmol eq of H⁺/g of catalyst C1 versus 1.1 mmol eq of H⁺/G of catalyst C2, whereby the proton exchange capacity of C1 and C2 is measured by potentiometry by neutralization of KOH). These results are explained by a better effectiveness of the acid functions of catalyst C1 relative to those of the catalyst C2, which while being less accessible to the ethyl tert-amyl ether, thus promote the secondary reactions at the expense of the desired primary reaction.

Without further elaboration, it is believed that one skilled in the art can, using the preceding description, utilize the present invention to its fullest extent. The preceding examples can be repeated with similar success by substituting the generically or specifically described reactants and/or operating conditions of this invention for those used in the preceding examples. Thus, the preceding preferred specific embodiments are, therefore, to be construed as merely illustrative, and not limitative of the remainder of the disclosure in any way whatsoever.

In the foregoing and in the examples, all temperatures are set forth in degrees Celsius and, all parts and percentages are by weight, unless otherwise indicated.

The entire disclosures of all applications, patents and publications, cited herein and of corresponding French application Ser. No. 08/02.800, filed May 23, 2008, are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

In the following claims the term “substantially spherical elementary particles” is intended to define the morphology of the particles as determined by one of ordinary skill in the art analyzing photographs obtained by scanning electron microscopy (SEM). In other words, the particle is not necessarily perfectly spherical, but instead is sufficiently spherical to be distinguished from other geometric shapes on the one hand, and which provides improved results on the other hand.

1. A process for cracking tert-alkyl ether(s) selected from among tert-amyl methyl ether (TAME) and ethyl tert-amyl ether (ETAE) for the production of tertiary olefins comprising bringing said tert-alkyl ether(s) into contact with at least one catalyst comprising at least one mesostructured hybrid organic-inorganic material that consists essentially of at least two substantially spherical elementary particles, whereby each of said substantially spherical particles consists essentially of a mesostructured matrix comprising a silicon oxide base to which are linked organic groups having terminal reactive acid functions, whereby said organic groups represent less than 20 mol % of said matrix present in each of said substantially spherical elementary particles, which substantially spherical particles have a maximum diameter of between 50 nm and 200 μm.

2. A process for cracking tert-alkyl ether(s) according to claim 1, wherein the mesopores of said mesostructured matrix have a diameter between 1.5 and 30 nm.

3. A process for cracking tert-alkyl ether(s) according to claim 1 wherein said acid terminal reactive functions of the organic groups that are linked to said mesostructured matrix are selected from among the following functions: sulfonic acid —SO₃H, carboxylic acid —COOH and functional acid derivative thereof, hydroxyl-OH, and phosphonic acid PO₃H.

4. A process for cracking tert-alkyl ether(s) according to claim 3 wherein said acid terminal reactive functions are sulfonic acid functions.

5. A process for cracking tert-alkyl ether(s) according to claim 1, wherein said organic groups represent 0.1 to 19.5 mol % of said matrix.

6. A process for cracking tert-alkyl ether(s) according to claim 1, wherein said silicon oxide-based mesostructured matrix is entirely silicic.

7. A process for cracking tert-alkyl ether(s) according to claim 1, wherein said silicon oxide-based mesostructured matrix comprises at least one element Z selected from aluminum, titanium, tungsten, zirconium and cerium.

8. A process for cracking tert-alkyl ether(s) according to claim 1, wherein said spherical elementary particles have a diameter of between 50 nm and 200 μm.

9. A process for cracking tert-alkyl ether(s) according to claim 1, wherein said mesostructured hybrid organic-inorganic material has a specific surface area of between 100 and 1500 m²/g.

10. A process for cracking tert-alkyl ether(s) according to claim 1, conducted under the following operating conditions: the temperature is between 100 and 200° C., the pressure is between 5 and 10·10⁵ Pa, and the VVH (hourly volume of feedstock related to the volume of catalyst) is between 4 and 40 h⁻¹.

11. A process for cracking tert-alkyl ether(s) according to claim 1, conducted in at least one reaction zone comprising at least one reactor that operates in a fixed bed, a moving bed, an expanded bed, or a fluidized bed.

12. A process according to claim 1, wherein the morphology of the substantially spherical elementary particles is determined by scanning electron microscopy (SEM).

13. A catalyst comprising at least one mesostructured hybrid organic-inorganic material that consists essentially of at least two substantially spherical elementary particles, whereby each of said substantially spherical particles consists essentially of a mesostructured matrix comprising a silicon oxide base to which are linked organic groups having terminal reactive acid functions, whereby said organic groups represent less than 20 mol % of said matrix present in each of said substantially spherical elementary particles, which substantially spherical particles have a maximum diameter of between 50 nm and 200 μm.
